(19) United States
(12) Patent Application Publication
(10) Pub. No.: US 2023/0129380 A1
(43) Pub. Date: Apr. 27, 2023

(54) Method for Estimating Molecular Complexity

(71) Applicant: The University Court of The University of Glasgow, Glasgow, Strathclyde (GB)

(72) Inventor: Leroy Cronin, Glasgow (GB)

(21) Appl. No.: 17/912,382

(22) PCT Filed: Mar. 19, 2021
(86) PCT No.: PCT/GB2021/050690

§ 371 Date: Sep. 16, 2022

(30) Foreign Application Priority Data
Mar. 19, 2020 (GB) 2003993.9

(51) Int. Cl. G01N 30/86; G01N 30/72; G01N 24/08

(52) U.S. Cl. CPC G01N 30/8631; G01N 30/72; G01N 30/8679; G01N 24/08

(57) ABSTRACT

The present invention provides a method for estimating the molecular complexity of a sample, for example to determine whether biotic or synthetic components are present in the sample. The method comprises the steps of (a) performing one of MS/MS, NMR or IR on a sample; (b) determining the unique peaks in the resulting spectrum; and (c) calculating the molecular assembly index of the sample based on the number of unique peaks in the spectrum.

### RELATED APPLICATION

The present application claims priority to, and the benefit of, GB 2003993.9 filed on 19 Mar. 2020 (19/03/2020), the contends of which are hereby incorporated by reference in their entirety.

### FIELD OF THE INVENTION

The present invention relates to a method for estimating the molecular complexity of a sample, such as an environmental sample, and the use of the method in the detection of life.

### BACKGROUND

A biosignatures is an object, substance or pattern whose origin specifically requires a biological agent (des Marais & Walter, des Marais). Biosignature candidates include atmospheric patterns such as changes in atmospheric composition associated with changing seasons (Walker, Schmieterman), variation in surface reflectance due to vegetation (Seager), specific biomolecules such as lipids (Georgiou & Deamer) or nucleic acids (Benner), homochiral polymers (Macdermott) and isotopic fractionation (Anbar).

Unfortunately, these biosignatures are associated with several downsides. Alien vegetation might not share the spectral characteristics of terrestrial plants. Similarly, relying on the detection of specific organic molecules prohibits us from detecting life based on undiscovered, extra-terrestrial biochemistry. Abiotic processes are known to produce enantiomeric excesses (Breslow & Levine), and isotopic fractionation can also be generated by non-living systems (Neveu). Moreover, effective evaluation of isotopic samples requires prior knowledge of the relevant metabolic pathways, again restricting the applicability to known, terrestrial life.

Living systems are able to generate complex molecules in a way that is not possible for abiotic systems. Similarly, humanity produces non-biological but highly complex molecules as a result of industrial or technological processes. Therefore, the present inventor believes that a complexity-based model is a promising prospect for use as a molecular biosignature. On this basis, a good molecular complexity measure for the purpose of life detection should satisfy three criteria.

First, the complexity measure needs to reflect the pathways by which complex molecules are formed in order to provide a distinction between those molecules which can form by random interaction, and those that require biological or technological influence to form.

Second, the complexity measure needs to be conceptually simple and intrinsic, with minimal external input required. It is not possible to account for all of the rules of chemistry, environmental conditions, and the many interactions that molecules can undergo without generating a complexity model that is too convoluted to use. Similarly, it is important to avoid imposing external weightings that do not necessarily correlate with the likelihood of abiotic formation, such as ring counts or the presence of specific functional groups or heteroatoms.

Finally, there must be a consistent experimental method for estimating the complexity measure so that unknown molecular samples can be analysed for signs of life.

The theory of molecular complexity has been extensively explored and many metrics have been devised based on structural, topological, or graphical complexity. These include measures based on specific graph features, such as counts of atoms/bonds (Minoli), distances between atoms in the molecular graph (Bonchev & Trinajstić; Balaban), paths through the molecular graph (Proudfoot) and total walk counts (Rücker & Rücker (2000)), connectivity of atoms (Randić & Pla\check{v}ić; Zhang), number of subgraphs (Rücker & Rücker (2001)), fractal dimensions (von Korff & Sande), and information theory based on molecular symmetry (Bonchev & Trinajstić; Bertz; Böttcher). Other complexity measures rely on weighting for specific molecular features such as the number of rings, heteroatoms, and properties such as electronegativity (Barone & Chanon; Allu & Oprea). Complexity measures have also been proposed which use machine learning (Coley; Li & Eastgate), and crowdsourcing (Sheridan).

The present inventor previously proposed a measure of molecular complexity known as object assembly (also known as pathway complexity) which is derived from the theory of assembly pathways (Marshall (2017)). Assembly pathways are sequences of joining operations that start with basic building blocks (e.g. bonds) and end with a final product. In these sequences, sub-units generated within the sequence can combine with other basic or compound sub-units later in the sequence to recursively generate larger structures (see FIG. 1).

Assembly pathways have been formalised mathematically using graph theory, using directed multigraphs (graphs where multiple edges are permitted between two vertices) with objects as vertices and objects as edge labels. The inventor's measure of molecular complexity, termed the molecular assembly index (MA), provides an agnostic measure of the likelihood for any molecular structure to be produced more than once. There will normally be multiple assembly pathways to create a given molecule. The molecular assembly index is the length of the shortest of those pathways. It is a simple integer metric to indicate the number of steps required in this idealized case to construct the molecule.

However, none of the molecular complexity measures proposed to date wholly fulfils the three criteria set out above. Most importantly, a consistent experimental method for estimating a complexity measure has not been described.

The present invention has been devised in light of these considerations.

### SUMMARY OF THE INVENTION

At its most general, the invention provides an experimental method for estimating molecular complexity. The method uses experimental techniques to assess the structural heterogeneity of a molecule within a sample, and provides an estimate of the molecular assembly index (MA) of the molecule.

Heterogeneous molecules typically have a varied composition (they contain many different chemical elements) and a varied structure (they contain many different substructures and lack symmetry). The heterogeneity of a molecule can be assessed using analytical experimental techniques, for example, by providing information on the quantity and identity of the different sub-structures within a molecule.

By calculating the molecular assembly index, the method relies on an intrinsic feature of a molecule, avoiding external input. The method reflects the pathways by which complex biotic or synthetic molecules are formed from simple precursors in contrast to abiotic molecules that can form by random interactions. More importantly, the method is agnostic and does not rely on assumptions based on recognised molecular structures, for example such as those derived from known, terrestrial biochemistry or from modern industrial manufacturing processes.

As such, the method allows the detection of complex molecules as biosignatures for extra-terrestrial life detection purposes. In the same way, the method allows the detection of terrestrial life, for example detection of organisms that live in extreme environments or for conformation that all microbes have been removed from a sterilised object. Also, as the complexity of a molecule is an important metric in designing therapeutic molecules, the method allows the assessment of potential candidate drug molecules.

Finally, the method has been validated on both single molecule samples and complex mixtures and it is shown to reliably detect complex molecules.

In a first aspect of the invention, there is provided a method for estimating the molecular complexity of a sample, the method comprising:
(a) performing one of MS/MS, NMR or IR on a sample;
(b) determining the unique peaks in the resulting MS2 spectrum for a parent ion in the MS1 spectrum, NMR spectrum, or IR spectrum; and
(c) calculating the molecular assembly index of the sample based on the number of unique peaks in the resulting MS2, NMR or IR spectrum.

The nuclear magnetic resonance (NMR) spectrum of a molecule is strongly influenced by the local magnetic environment around each of the observed atoms. If the local environment of two atoms is identical, the atoms appear at the same chemical shift and display the same coupling relationship. Conversely, if the observed atoms exist in different local magnetic environments, the atoms will display different chemical shifts and coupling patterns. Thus, NMR can provide information on the symmetry and hence heterogeneity of the molecule.

Similarly, the infrared (IR) spectrum of a molecule is influenced by the functional groups found within the molecule, as well as its overall structure. Molecules comprising multiple different functional groups will display many different peaks in their infrared spectrum.

Similarly, tandem mass spectrometry (MS/MS) provides information on the different fragments (substructures) within a molecule that correlates with the heterogeneity of the molecule.

The inventor has found that the number of peaks in the MS2, NMR and IR spectrum of a molecule is correlated with the MA index of the molecule. Thus, by using easily observable molecular properties, the method allows the detection of high MA molecules which do not form in the absence of biological or technological influence.

In a preferred embodiment of the invention, the method comprises performing tandem mass spectrometry on a sample. Thus, there is provided a method for estimating the molecular complexity of a sample, the method comprising:
(a) performing MS/MS on the sample;
(b) determining the unique peaks in an MS2 spectrum for a parent ion in the MS1 spectrum; and
(c) calculating the molecular assembly index of the sample based on the number of unique peaks in the MS2 spectrum.

Tandem mass spectrometry is preferred because it generates separate signals for different ions within a complex mixture. Thus, the method can be performed on a complex mixture of molecules of interest.

Step (a) comprises selecting an ion in the MS1 spectrum (a parent ion) for fragmentation. The parent ion may have a mass of 250 Da or more. Molecules having a mass of 250 Da or more display large compositional and structural diversity and so display a diverse range of MA values. As such, a suitable biosignature is likely to be found above this mass range.

The selected parent ion may have a mass of 1000 Da or less. The investors have found that sufficient complexity information can be derived from ions below 1000 Da.

The sample may comprise a single molecule of interest. In such cases, the estimated MA value for the sample directly corresponds to the estimated MA value for the molecule of interest.

Alternatively, the sample may comprise multiple different molecules of interest. In such cases, the estimated MA value for the sample may be calculated from the parent ion having the largest number of unique peaks in the MS2 spectrum.

In a second aspect, the invention provides a method for the detection of life, the method comprising:
(a) estimating the molecular complexity of a sample using the method of the first aspect; and
(b) comparing the calculated molecular assembly index of the sample to a threshold value.

The inventor has found that molecules possessing an MA value greater than 15 are likely to result from biological or technological processes. Thus, detection of molecules having an MA greater than 15 provides an agnostic method for detecting life, for example extra-terrestrial life.

The method is robust against false positives because the number of synthetic steps required to create a specific molecule in practice is unlikely to be lower than the number of steps predicted by the assembly model for that molecule. This provides greater confidence that molecules meeting the threshold are truly of biological or technological origin.

Moreover, by using simple experimental techniques such as mass spectrometry, and without the need for complex sample preparation, the method is suitable for use on space exploration craft, such as space probes or planetary rovers.

In a third aspect, the invention provides a method for identifying a candidate pharmaceutical or agrochemical molecule, the method comprising:
(a) estimating the molecular complexity of a molecule in a molecular library using the method of the first aspect; and
(b) selecting a molecule having a molecular assembly index greater that a threshold value as a candidate pharmaceutical or agrochemical molecule.

The complexity of a molecule is an important metric in designing bioactive molecules such as agrochemicals or pharmaceuticals. Thus, an experimental method for determining complexity can provide a simple and effective method for screening drug libraries for candidate molecules. Moreover, an experimental method for determining complexity can effectively screen samples comprising multiple different molecules, such as extracts from biological sources. Samples meeting the complexity threshold can then be selected for further analysis, and for purification and separation of their constituents.

These and other aspects and embodiments of the invention are described in further detail below.

### SUMMARY OF THE FIGURES

The present invention is described herein with reference to the figures listed below.

FIG. 1 depicts an assembly space for a target object that can be created from grey and white blocks. Some arrows have been omitted for clarity. The label on the arrow represents the object in the space that needs to be combined with the source to make the target. The dashed region represents an assembly subspace, which is the smallest subspace that contains the object made of a row of 4 grey blocks. The assembly index of that object is the number of objects in that subspace, not including basic objects.

FIG. 2 is a flow diagram describing an algorithmic implementation of a split-branch assembly index calculation as applied to molecular structures. The highlighted box corresponds to a subprocess described in FIG. 3.

FIG. 3 is a flow diagram describing the “Calculate Substructure MA” subprocess highlighted in the split-branch algorithm method of FIG. 2. The highlighted box corresponds to a subprocess described in FIG. 4.

FIG. 4 explains the “Calculate MA of Partition” subprocess highlighted in the split-branch algorithm method of FIG. 3. The highlighted box corresponds to a subprocess described in FIG. 3.

FIG. 5 provides examples of the split-branch calculation of molecular assembly index for tryptophan and penicillin.

FIG. 6 depicts a model of the assembly process as a random walk on weighted trees where the number of outgoing edges (leaves) grows as a function of the depth of the tree, due to the addition of previously made sub-structures. By generating several million trees and calculating the likelihood of the most likely path through the tree it is possible to estimate the likelihood of an object forming by chance as a function of the number of joining operations required (path length).

FIG. 7 shows choice distributions for various number of choices (k) and heterogeneity (h) values for a model of the assembly process as a random walk on weighted trees.

FIG. 8 shows the probability of the most likely path through a tree as a function of the path length, which decreases rapidly and quickly approaches 1 in 10²³ (dashed line). The uppermost line correspondents to α=2, the central line α=2.5 and the lowermost line α=3.

FIG. 9 shows the total number of possible hydrocarbon structural isomers up to 12 C atoms.

FIG. 10 shows the total number of possible structural isomers in molecules up to 10 atoms of C, N, O and S (excluding hydrogen atoms).

FIG. 11 shows the computed MA of molecules in the Reaxys database by molecular weight. The shading indicates the frequency of a molecule in a given molecular weight range with a given MA. The MA of 2.5 million molecules was calculated and the data subsampled to control for bias as explained below. The molecular masses are binned in 50 Da sections, and each molecular mass bin is normalized such that the total frequency of each molecular mass section is one.

FIG. 12 shows the observed correlation between the number of peaks in the MS2 fragmentation spectrum and the MA value of the ion for 116 common molecules. The shaded region shows the 90% prediction interval using quantile regression, with the median prediction shown in the centre line. The circles represent small organics while triangles represent peptide.

FIG. 13 shows three example molecular structures with associated MA index (A) and the fragmentation spectra associated with the respective molecular ions (B). The high MA molecules have more peaks in their fragmentation spectra. (C) to (E) depict the analytical workflow for measuring MA in mixtures. A single ion is selected based on intensity (C), and the MS2 spectra is recorded, with the inset showing the same spectra zoomed in on the shaded region to show lower intensity peaks (D). Many ions from the mixture are fragmented and the highest measured MA value represents the MA of the mixture (E).

FIG. 14 shows the predicted MA against the parent mass of many ions for different laboratory and environmental samples: (A) the predict MA for samples prepared in the laboratory; (B) the predicted MA for the laboratory samples in (A) separated by sample. It can be seen that the predicted MA from biological samples is clearly higher than abiotic or dead samples; (C) the predicted MA for samples collected from the environment; (D) the predicted MA for environmental the samples in (C), separated by sample.

FIG. 15 shows the observed correlation between the number of peaks in the MS2 spectrum and the MA value of a molecule.

FIG. 16 shows the observed correlation between the number of peaks in the NMR spectrum and the MA value of a molecule. The number of peaks in the NMR spectrum is calculated as the number of different peaks for carbon (C) and hydrogen (H) weighted against the number of C and CH moieties per molecule.

FIG. 17 shows the observed correlation between the number of peaks in the fingerprint region of the IR spectrum and the MA of a molecule. IR peals are calculated in the gas phase using ADF.

### DETAILED DESCRIPTION OF THE INVENTION

The present invention provides an experimental method for estimating molecular complexity. The method uses simple experimental techniques such as MS/MS, NMR or IR to estimate the molecular assembly index (MA) of a sample.

It is noted that the use of MALDI-TOF mass spectra for quantifying biological samples using linear passion independent component analysis is described by Deepaisarn et al.

Sample

The sample may comprise a single molecule of interest. That is, a single molecule for which the complexity measurement is to be conducted. The sample may comprise additional molecules which are not of interest, for example solvent or other additives introduced during sample gathering and preparation.

The identity of the molecule of interest may be known (for example, where the sample is prepared from a known material) or unknown (for example, where the sample is prepared by separating the components of an unknown environmental sample).

The sample may comprise multiple molecules of interest. That is, the sample may be a mixture of molecules. The identity of the component molecules may be known or unknown.

The sample may be an environmental sample. That is, a sample collected from the environment.

The sample may be an extract from a biological source (e.g. an extract from a fermentation broth, or a plant extract).

The sample may be from an extra-terrestrial source.

The sample may be dissolved in a suitable solvent. The sample may be filtered prior to analysis. Typically, the sample does not require further purification, for example chromatographic separation. This simplifies the procedure.

Mass Spectrometry Measurement

The method for estimating molecular complexity may comprise performing tandem mass spectrometry on a sample. That is, the method may comprise:
(a) performing MS/MS on a sample;
(b) determining the unique peaks in an MS2 spectrum for a parent ion in the MS1 spectrum; and
(c) calculating the molecular assembly index of the sample based on the number of unique peaks in the MS2 spectrum.

In tandem mass spectrometry (MS/MS), a first mass spectrum (MS1) of a sample is recorded. Then, a single ion (parent ion) from the first mass spectrum is selected for fragmentation. After fragmentation, a second mass spectrum (MS2) of the fragments is recorded.

An advantage of tandem mass spectrometry is that it generates separate signals for different ions within a complex mixture. Thus, the method can be performed on a complex mixture of molecules of interest.

Suitable tandem mass spectrometry configurations include triple quadrupole (TQ), quadrupole time-of-flight (Q-TOF), ion trap time-of-flight (IT-TOF), quadrupole ion trap (Q-IT), quadrupole ion-cyclotron-resonance (Q-ICR), ion trap ion-cyclotron resonance (IT-ICR), ion trap orbitrap (IT-Orbitrap), double time-of-flight (TOF-TOF) and multistage MS (MSn).

Suitable fragmentation methods include in-source fragmentation, collision-induced dissociation (CID), electron transfer dissociation (ETD), electron capture dissociation (ECD), photodissociation and surface-induced dissociation. Preferably, CID is used. Suitable CID methods include low-energy CID and high-energy CID (HECID), for example higher-energy collisional dissociation (HCD) also known as higher-energy C-trap dissociation. Preferably, HCD is used as it is able to resolve ions of lower molecular mass, such as ions in the range 200 to 1000 Da.

Parent Ion Selection

The method for estimating molecular complexity using MS/MS comprises selecting an ion in the MS1 spectrum for fragmentation. This selected ion is called the parent ion.

Where the sample comprises a known molecule, the parent ion can be appropriately selected in the MS1 spectrum. That is, the parent ion can be selected to correspond to the mass of the molecular ion. Typical positive molecular ions take the form M⁺, [M+H]⁺ or [M+X]⁺, where X is a cationic species, such as an alkali metal cation. Typical negative molecular ions take the form M⁻, [M−H]⁻ or [M+Y]⁻, where Y is an anionic species, such as a formate anion.

Where the sample comprises an unknown molecule, or where the sample comprises multiple molecules, the parent ion can be selected in the MS1 spectrum based on the observed mass.

Preferably, the method comprises selecting a parent ion in the MS1 spectrum having a minimum mass of 200 Da or more, more preferably 250 Da or more, even more preferably 275 Da or more and most preferably 300 Da or more. The inventor has found that molecules having this minimum mass show a diverse range of MA values and so high MA molecules arising from biological or technological processes are likely to meet this minimum value.

Preferably, the method comprises selecting a parent ion in the MS1 spectrum having a maximum mass of 1000 Da or less, more preferably 800 Da or less, even more preferably 600 Da or less and most preferably 500 Da or less.

The inventor has found that sufficient information can be obtained when the selected parent ion has a mass that is in a range selected from the upper and lower amounts given above, for example in the range 200 to 1000 Da, such as 250 to 800 Da or 300 to 500 Da.

Where the sample contains an unknown molecule, or where the sample contains multiple molecules, multiple parent ions can be selected in the MS1 spectrum.

Typically, the most intense parent ions in the MS1 spectrum are selected. That is, the parent ions having the largest intensity (counts per second, cps) are selected.

Preferably, the method comprises selecting 30 or fewer parent ions in the MS1 spectrum, more preferably 25 or fewer parent ions, even more preferably 20 parent ions or fewer and most preferably 15 or fewer parent ions are selected in the MS1 spectrum. Limiting the number of parent ions selected for fragmentation and analysis reduces the time required for analysis and improves the efficiency of the method.

Preferably, the method comprises selecting 2 or more parent ions in the MS1 spectrum, more preferably 3 or more ions, even more preferably 4 or more ions and most preferably 5 or more ions.

The number of ions selected in the MS1 spectrum can be in range selected from the upper and lower amounts given above, for example in the range 2 to 20 ions, such as 5 to 20 ions or 10 to 15 ions.

Typically, the method comprises temporarily excluding from analysis those parent ions which have recently been fragmented and analysed to provide an MS2 spectrum. This maximises the number of parent ions that can be selected and analysed. Methods of temporarily excluding parent ions from analysis are known, and include Data Dependent Acquisition (DDA). In such methods, a dynamic exclusion window is used to ensure that parent ions appearing multiple times in a set time interval are excluded from the analysis for a certain time period. The relevant time periods can be appropriately adjusted. Typically, the parent ions appearing twice in a 5 to 30 second interval are excluded, such as parent ions appearing twice in 10 seconds. Typically, such parent ions are excluded from analysis for a period ranging from 10 to 90 seconds, for example 20 seconds, 30 seconds or 45 seconds.

MS2 Peak Determination

The method for estimating molecular complexity using MS/MS comprises determining the unique peaks in an MS2 spectrum for a parent ion in the MS1 spectrum. Unique peaks are those peaks having a unique mass and which correspond to distinct fragments of the parent ion.

Typically, all peaks in the MS2 spectrum are counted. Then, the peaks are filtered to arrive at the number of unique peaks. That is, certain peaks in the MS2 spectrum are either disregarded or merged (combined) with adjacent peaks.

Typically, the method comprises disregarding peaks in the MS2 spectrum having a maximum intensity below a certain threshold.

The threshold can be determined based on the spectrometer. For example, the method may comprise disregarding all peaks having a maximum intensity of 10,000 cps or less, more preferable 20,000 cps or less, even more preferably 40,000 cps or less and most preferably 50,000 cps or less.

The threshold can be determined based on the observed MS2 spectrum. For example, the method may comprise disregarding all peaks having a maximum intensity of 0.5% or less relative to the highest recorded intensity in the MS2 spectrum, such as 1.0% or less or 2.0% or less.

Typically, the method comprises merging all peaks having an observed mass within a certain distance of an adjacent peak in the MS2 spectrum. Merging of peaks can be appropriately done based on standard methods. For example, the largest local peak (local maximum) can be chosen and the peaks within a given distance merged (discounted) with that peak. The process can be repeated until suitable spectra have been recorded.

Merging of peaks can be appropriately selected based on the resolution of the mass spectrometer. Preferably, the method comprises merging all peaks within ±0.005 Da of an adjacent peak in the MS2 spectrum, more preferably the method comprises merging all peaks within ±0.01 Da of an adjacent peak in the MS2 spectrum.

Different measures of molecular assembly index are known. The measure of molecular assembly index used in the worked examples does not include hydrogen atoms. In such a case, the inventor additionally merges any peak within ±1.0 Da of an adjacent peak in the MS2 spectrum.

One MS2 spectra per parent ion may be recorded. Typically, the method comprises repeating the measurement to generate multiple MS1 and MS2 spectra. In such cases, peaks not appearing in a certain proportion of the MS2 spectra corresponding to the same parent ion can be disregarded. Thus, the analysis removes inconsistent peaks. This improves reproducibility.

Preferably, the method comprises disregarding all peaks present in fewer than 10% of the MS2 spectra for a selected parent ion, more preferably fewer than 15%, even more preferably fewer than 20% and most preferably fewer than 25% of the MS2 spectra for a selected parent ion.

The number of peaks in the MS2 spectrum may be adjusted based on the observed peaks in the MS1 spectrum. Optionally, the number of peaks in the MS2 spectrum may be divided by the number of peaks found within 0.5 Da of the parent mass in the MS1 spectrum. Where a sample comprises multiple molecules, this adjustment compensates for co-fragmentation and merging of different MS1 parent ions into the same MS2 spectra.

Where a sample comprises multiple molecules, additional peak filtering may be used to account for excessive number of ions in the spectra. For example, the method comprises disregarding all peaks having a relative intensity below a certain fraction of the most intense peak in the MS2 spectrum. Typically, the method comprises disregarding all peaks having a relative intensity below 2% of the highest recorded intensity in the MS2 spectrum, such as below 5% or below 10%.

NMR Measurement

The method for estimating molecular complexity may comprise performing nuclear magnetic resonance (NMR) spectroscopy on a sample. That is, the method may comprise:
(a) performing NMR spectroscopy on a sample;
(b) determining the unique peaks in the resulting NMR spectrum; and
(c) calculating the molecular assembly index of the sample based on the number of unique peaks in the resulting NMR spectrum.

In NMR spectroscopy, a particular spin-active nucleus is selected for observation. The observed chemical shifts in the resulting NMR spectrum indicate the different unique magnetic environments within the molecule, and the observed intensity of each peak indicates the relative occurrence of each of those environments within the molecule.

An advantage of NMR spectroscopy is that is a non-destructive technique. Thus, sample can be recovered after measurement of the NMR spectrum.

Suitable spin active nuclei include ¹H, ¹¹B, ¹³C, ¹⁷O, ¹⁹F, ²⁹Si and ³¹ P. Due to their relative abundance and ease of analysis, NMR based on ¹H and ¹³C is preferred.

The method comprises determining the unique peals in the resulting NMR spectrum. Typically, all peaks in the NMR spectrum are counted. Then, the peaks are weighted to arrive at the number of unique peaks.

Typically, the method comprises counting all peaks in the ¹H and ¹³C NMR for a given molecule and then weighting this value against the number of C and CH moieties per molecule.

IR Measurement

The method for estimating molecular complexity may comprise performing infrared spectroscopy on a sample. That is, the method may comprise:
(a) performing IR spectroscopy on a sample;
(b) determining the unique peaks in the resulting IR spectrum; and
(c) calculating the molecular assembly index of the sample based on the number of unique peaks in the resulting NMR spectrum.

In IR spectroscopy infrared light interacts with (is absorbed by) a molecule. The frequency of the absorbance corresponds to the molecular vibrational or rotational modes of the molecule. This is dependent on the underlying structure of the molecule, include the different functional groups or bonds found within a molecule and its symmetry.

An advantage of IR spectroscopy is that it is a non-destructive technique. Thus, a sample can be recovered after measurement of the IR spectrum.

The method comprises determining the unique peaks within a certain frequency window in the IR spectrum.

Preferably, the method comprises determining the unique peaks with a maximum frequency below 4,000 cm⁻¹, more preferably below 2,500 cm⁻¹, even more preferably below 2,000 cm⁻¹ and most preferably below 1,500 cm⁻¹.

Preferably, the method comprises determining the unique peaks with a minimum frequency above 0 cm⁻¹, more preferably above 50 cm⁻¹ and even more preferably above 100 cm⁻¹.

The inventor has found that sufficient information can be obtained when the unique peaks have a frequency that is in a range selected from the upper and lower amounts given above, for example in the range 0 cm⁻¹ to 4,000 cm⁻¹, such as 0 cm⁻¹ to 1,500 cm⁻¹ or 100 cm⁻¹ to 1,500 cm⁻¹. Specifically, the inventor has found that the number of peaks within the fingerprint region of the IR spectrum is correlated with the structural heterogeneity of a molecule and the molecular assemble index.

Calculating Molecular Assembly Index

The method for estimating molecular complexity comprises calculating the molecular assembly index based on the number of unique peaks in an MS2, NMR or IR spectrum. The molecular assembly index is a simple integer measure that describes the complexity of a molecule. It indicates the minimum number of steps required to construct the molecule from basic building blocks (Marshall (2019)).

The molecular assembly number is proportional to the number of unique peaks in the MS2, NMR or IR spectrum. Thus, by scaling the number of unique peaks in the MS2, NMR or IR spectrum, it is possible to determine the MA index of the molecule. Scaling (normalizing) the number of unique peaks to obtain the MA index of the molecule allows the values obtained from different experimental methods to be appropriately compared.

Typically, the molecular assembly number is directly proportional with the number of unique peaks in the MS2, NMR or IR spectrum. Thus, the molecular assembly number (MA) can be calculated by scaling the number of unique peaks (x) by a certain magnitude (m). That is, the molecular number can be calculated using the equation:

MA=m(x)

Typically, the molecular assembly number is linearly correlated with the number of unique peaks in the MS2, NMR or IR spectrum. Thus, the molecular assembly number (MA) can be calculated by adding an off-set (c) to the number of unique peaks (x) after scaling by a magnitude (m). That is, the molecular assembly number can be calculated using the equation:

MA=m(x)+c

Where the molecular assembly number (MA) is calculated based on the number of unique peaks in the MS2 spectrum (x_{MS2}), the value of m is typically in the range 0.3 to 0.7. Preferably, m is in the range 0.4 to 0.6, more preferably, 0.45 to 0.55.

Where the molecular assembly number (MA) is calculated based on the number of unique peaks in the MS2 spectrum (x_{MS2}), the value of c is typically in the range 4 to 9. Preferably, c is in the range 5 to 8, more preferably 6 to 7.

Most preferably, where the molecular assembly number (MA) is calculated based on the number of unique peaks in the MS2 spectrum (x_{MS2}), the molecular assembly number (MA) is calculated using the equation:

MA=0.48(x_{MS2})+6.58

Where the molecular assembly number (MA) is calculated based on the number of unique peaks in the NMR spectrum (x_{NMR}), the value of m is typically in the range 2.5 to 5. Preferably, m is in the range 3 to 4, more preferably, 3.5 to 3.6.

Where the molecular assembly number (MA) is calculated based on the number of unique peaks in the NMR spectrum (x_{NMR}), the value of c is typically in the range 0 to −7. Preferably, c is in the range −1 to −6, more preferably −2 to −5.

Most preferably, where the molecular assembly number (MA) is calculated based on the number of unique peaks in the NMR spectrum (x_{NMR}), the molecular assembly number (MA) is calculated using the equation:

MA=3.55(x_{NMR})−3.41

Where the molecular assembly number (MA) is calculated based on the number of unique peaks in the IR spectrum (x_{IR}), the value of m is typically in the range 0.3 to 0.7. Preferably, m is in the range 0.4 to 0.6, more preferably, 0.45 to 0.55.

Where the molecular assembly number (MA) is calculated based on the number of unique peaks in the IR spectrum (x_{IR}), the value of c is typically in the range 4 to 9. Preferably, c is in the range 5 to 8, more preferably 6 to 7.

Most preferably, where the molecular assembly number (MA) is calculated based on the number of unique peaks in the IR spectrum (x_{IR}), the molecular assembly number (MA) is calculated using the equation:

MA=0.51(x_{MS2})+6.60

Additional Methods

An experimental method for measuring molecular complexity has several technical applications.

Highly complex molecules are produced by biological or technological processes, and so the presence of a highly complex molecule can indicate the existence of a biological or technological process. Thus, highly complex molecules can act as an indicator of life (e.g. as a biosignature). Experimental determination of highly complex molecules can find use in life detection.

Thus, the invention also provides a method for the detection of life, the method comprising:
(a) estimating the molecular complexity of a sample according to any method described herein; and
(b) comparing the calculated molecular assembly index to a threshold value.

The sample may be a sample of extra-terrestrial material, for example, a sample of extra-terrestrial water, ice, rock or other minerals. In such cases, the method may be used to detect extra-terrestrial life.

The sample may be a sample of terrestrial material, for example, a sample of terrestrial soil, water, ice or geological material. In such cases, the method may be used to find life in extreme conditions (extremophiles).

The sample may be a sample of material that has undergone a sterilisation procedure, such as heat-based sterilisation (e.g. autoclaving or incineration), chemical-based sterilisation (e.g. treatment with bleach or ozone) or radiation-based sterilisation (e.g. treatment with ultraviolet light or ionising radiation). In such cases, the method may be used to detect if a sample has been appropriately cleaned, for example, to remove microbes.

The threshold value for molecular assembly index may be appropriately chosen depending on the intended use. In the case of life detection, the threshold value for molecular assembly index may be at least 12, such as at least 15 or at least 20.

Alternatively, the threshold value may be determined based on known molecules. For example, a set of known molecules of biological or technological origin (a training set) may be used to determine the threshold value. For example, the theoretical IR of NMR spectrum of the set of known molecules may be calculated using known techniques (e.g. density functional theory) and an appropriate threshold values chosen based on the results. Alternatively, the MA of molecules in the training set can be experimentally measured using the methods described above and an appropriate threshold value chosen. Similarly, the MA values could be theoretically calculated and an appropriate threshold value chosen.

That is, the method may comprise:
(i) providing a training set comprising one or more molecules of biological and technological origin; and
(ii) determining the average molecular assembly index for the molecules in the training set.

The complexity of a molecule is correlated with the biological activity of a molecule (Hann). Thus, identifying highly complex molecules is useful in providing candidate molecules for pharmaceutical or agrochemical development.

Thus, the invention also provides a method for identifying candidate pharmaceutical or agrochemical molecules, the method comprising:
(a) estimating the molecular complexity of a sample in a molecular library using any method described herein; and
(b) selecting a sample having a molecular assembly index greater that a threshold value as a candidate pharmaceutical or agrochemical molecule.

The molecular library may comprise known molecules. Alternatively, the molecular library may comprises unknown molecules. Similarly, the molecular library may comprise mixtures of molecules, such as extracts from biological sources (e.g. fermentation broth, plant extracts). Thus, the method allows mixtures to be screened and those mixtures comprising complex molecules can be rapidly identified and selected for further analysis.

The selected sample can be further analysed to determine the contents. For example, the constituents may be isolated (separated) and/or purified. That is, the method may further comprise:
(c) separating the sample constituents.

Optionally, the method may comprises screening the sample constituents for biological activity. That is, the method may further comprise:
(d) screening the sample constituents for biological activity.

The threshold value may be appropriately chosen based on known pharmaceutical or agrochemical molecules. For example, the threshold value for molecular assembly index may be at least 12, such as at least 15 or at least 20.

Alternatively, the threshold value may be determined based on known molecules. For example, a set of known molecules displaying a target bioactivity (a training set) may be used to determine the threshold value. For example, the theoretical IR of NMR spectrum of the set of known molecules may be calculated using known techniques (e.g. density functional theory) and an appropriate threshold values chosen based on the results. Alternatively, the MA of molecules in the training set can be experimentally measured using the methods described above and an appropriate threshold value chosen. Similarly, the MA values could be theoretically calculated and an appropriate threshold value chosen.

That is, the method may comprise:
(i) providing a training set comprising one or more molecules displaying a target bioactivity; and
(ii) determining the average molecular assembly index for the molecules in the training set.

The average molecular assembly index can be used as the threshold value in the method for identifying a candidate pharmaceutical or agrochemical molecule. Alternatively, the threshold value can be selected to be slightly below the average molecular assembly index (for example, one or two units below the average molecular assembly index).

Computer Program

The invention also comprises computer-implemented methods and computer programs for estimating the molecular complexity of a sample.

The invention provides a computer-implemented method for estimating the molecular complexity of a sample, the method comprising the steps of:
(a) receiving MS/MS, NMR or IR data in respect of a sample;
(b) determining the unique peaks the MS2, NMR or IR spectrum; and
(c) calculating the molecular assembly index of the sample based on the number of unique peaks in the MS2, NMR or IR spectrum.

Typically, step (b) comprises counting all peaks in the MS2, NMR or IR spectrum. Then, the peaks are filtered to arrive at the number of unique peaks. That is, certain peaks in the MS2, NMR or IR spectrum are either disregarded or merged (combined) with adjacent peaks. Suitable methods for disregarding or merging the peaks include the methods set out in the section entitled “unique peaks” above.

Typically, step (c) comprises scaling the number of unique peaks by a certain magnitude, and optionally adding an off-set. Suitable methods for calculating the molecular assembly index based on the number of unique peaks in the MS2, NMR or IR spectrum are set out in the section entitles “calculating molecular assemble number” above.

The invention also provides a data processing device comprising:
(a) means for receiving MS/MS, NMR or IR data in respect of a sample;
(b) means for determining the unique peaks in an MS2, NMR or IR spectrum; and
(c) means for calculating the molecular assembly index of the sample based on the number of unique peaks in the MS2, NMR or IR spectrum.

The invention also provides a computer program comprising instruction which, when the program is executed on a computer, cause the computer to carry out the steps of:
(a) receiving MS/MS, NMR or IR data in respect of a sample;
(b) determining the unique peaks in an MS2, NMR or IR spectrum; and
(c) calculating the molecular assembly index of the sample based on the number of unique peaks in the MS2, NMR or IR spectrum.

The invention also provides a computer-readable storage medium comprising instructions which, when executed by computer, cause the computer to carry out the steps of:
(a) receiving MS/MS, NMR or IR data in respect of a sample;
(b) determining the unique peaks in an MS2, NMR or IR spectrum; and
(c) calculating the molecular assembly index of the sample based on the number of unique peaks in the MS2, NMR or IR spectrum.

That is, the invention also provides a computer-readable storage medium having stored thereon any computer program disclosed herein.

### OTHER EMBODIMENTS

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above.

Calculated MA

Relevant aspects of the general theory of object assembly are described below, along with the theory of molecular assembly and the model of molecular synthesis used to calculate the molecular assembly index. Concepts related to Assembly Spaces and the Assembly Index are formalised in Marshall (2019).

Theory of Object Assembly

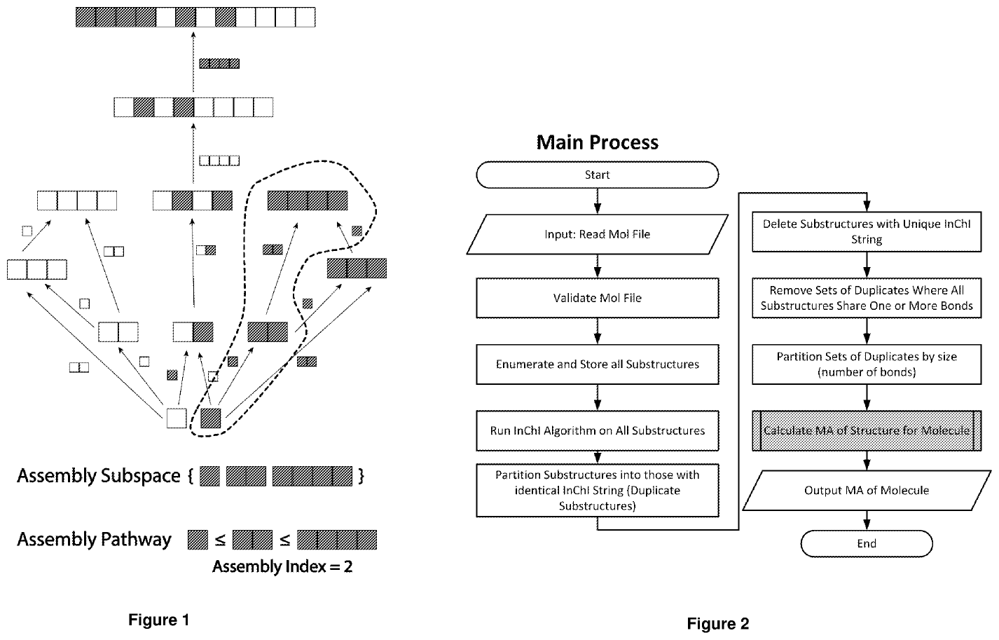

The Object Assembly Index (OA) of an object is defined in the context of an assembly space (Marshall (2019)), which defines how objects can be made from a set of basic building blocks through combination operations. Each point in the assembly space is an object, and arrows between objects A and C are labelled by another object B with the implication that A and B can be combined in some predetermined way to make object C (FIG. 1). There is a symmetric arrow in the space between objects B and C, labelled with object A. Traversal along arrows in the assembly space represents a series of joining operations. An Assembly Subspace is a subset of objects and arrows that itself constitutes an assembly space. A subspace that contains the irreducible building blocks of the parent assembly space (a subspace that is “rooted”) and a target object X can be thought of as containing a recipe to create object X using joining operations. The OA of X is defined as the size of the smallest rooted Assembly Subspace containing X. The OA can be thought of as the minimum number of joining operations required to create X, starting from basic objects, where objects created in the initial steps can be “re-used” in subsequent joining operations.

Intuitively, the OA of an object is correlated positively with its size, and negatively with the number of repeated and non-overlapping substructures along the minimal pathways. Any such substructures could themselves contain repeated substructures, further reducing the OA, recursively. Objects with low OA are those objects which are small and/or contain internal symmetries, while objects with high OA tend to be large and heterogenous. An upper bound for the OA of an object of size s is s−1, based on the fact that it is always possible to construct an object by adding a single basic object at each step. A lower bound can be found by considering that at each step it is possible to join the object created in the previous step to itself, and an object created this way in n steps will have size s=2ⁿ, with n being the minimum possible OA of an object of size s. Therefore, log₂ s is a lower bound for the OA of any object of size s.

Construction of an object using the object assembly model is designed to mimic the construction of objects through random collisions starting from basic building blocks, and determining the shortest pathway indicates the minimum number of steps that are needed for construction. Thus, the model provides a lower bound on the probability of an object forming in comparison to all other objections that can be created through undirected (random) interaction.

Theory of Molecular Assembly

Object Assembly theory can be applied to molecules and the present model was devised with molecules in mind. It is possible to use either atoms or bonds as the irreducible objects. Here, a graph-theory based model that considers only the connectivity of atoms and bonds within molecules, restricted by normal valence rules, is used. Hydrogen depleted representations are used to reduce computational cost.

Assembly pathways in the present model are not representative of real molecular synthesis but rather represent a synthesis in which all the complexities of chemistry, other than valence rules, are ignored. Considering a syntheses in which all steps take the form A+B→C, where C is the only product, then the space of synthetic pathways of this type is an Assembly Subspace of the Assembly Space used in the present model, containing a subset of the structures and connections between them. The inventor has previously shown that the MA in an Assembly Subspace is an upper bound for the MA in the original space, and hence such synthetic pathways cannot be shorter (Marshall (2019)). In cases where A+B→C+D, or A→C+D, the most complex product will tend to have lower MA than in the case of A+B→C. Therefore, such steps tend to result in longer synthetic pathways rather than shorter ones. Since most steps in the present model represent highly simplified synthetic steps, the MA of a molecule provides a reasonable lower bound on the shortest synthetic pathway from atomic starting materials.

It is possible for a molecule to have a low MA yet require a much larger number of synthetic steps to construct in practice. In the context of life detection, this could result in false negatives. However, this measure is robust against false positives. This provides greater confidence in the biological or technological origin of the detected molecules.

The present model calculates a variant of the Object Assembly Index known as the split-branched object assembly index. This variant was chosen for algorithmic simplicity (Marshall (2019)). The split-branch object assembly index of a molecule is an upper bound for the MA of the molecule (Marshall (2019)), although there is an offset of 1 between the variants as the initial step of the assembly index is a joining of two basic objects whereas the initial step of the split-branch process can be thought of as laying down a single basic object. The split-branch variant can be considered intuitively as forming structures in their own separate environments before bringing them together. Therefore, a substructure used to create one object cannot be used to create a separate object without rebuilding the substructure. (In a conventional OA measure, one can think of all the structures forming in the same environment, and such reuse would be permitted.) For simplicity, “MA” is used to mean the split-branched pathway assembly complexity herein.

The present model calculates the MA on hydrogen depleted graphs using bonds as basic objects. This reduces computational complexity and allows for simpler representation of molecular fragments. A molecular graph is selected and all possible connected substructures are calculated then grouped into identical fragments. This grouping is done by associating fragments with their InChI string, using the InChI API (Heller) as the InChI string is a canonical representation of a chemical structure (i.e. each chemical structure is represented by a single unique InChI string). The MA is determined by searching through partitions of identical (non-overlapping) substructures, with each unique substructure in a molecule contributing its own MA to the MA of the target, plus 1 for each time it is duplicated. The MA of the substructures is calculated recursively, unless it can be determined implicitly due to the substructure size being 3 bonds or fewer (substructures of size 1, 2, and 3 bonds have MA 1, 2, and 3 respectively).

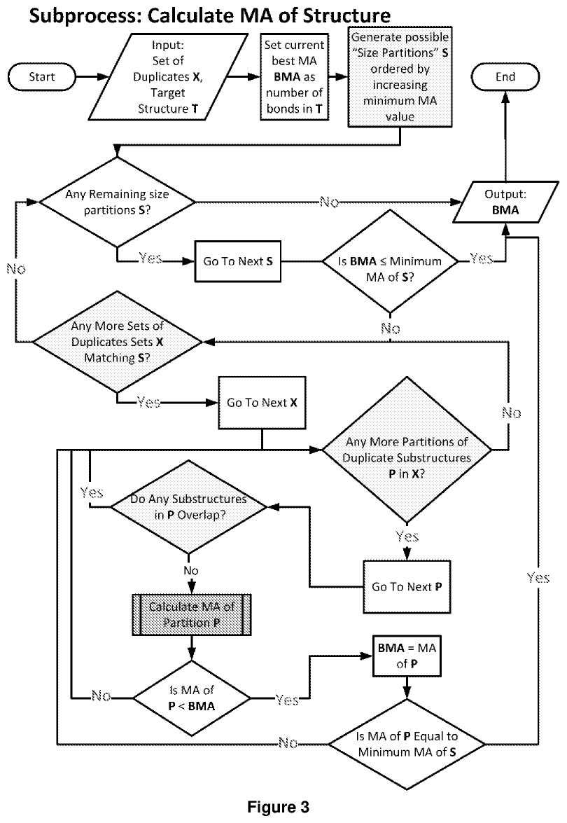

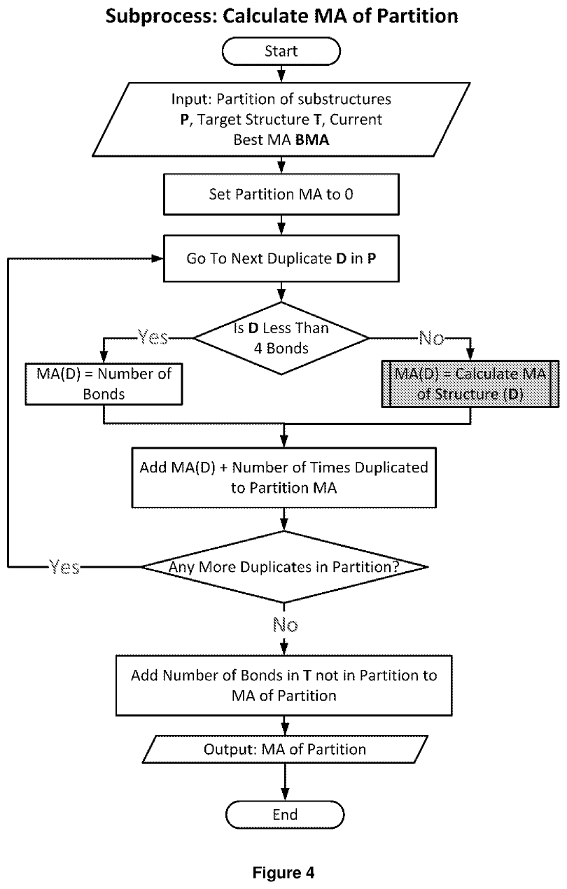

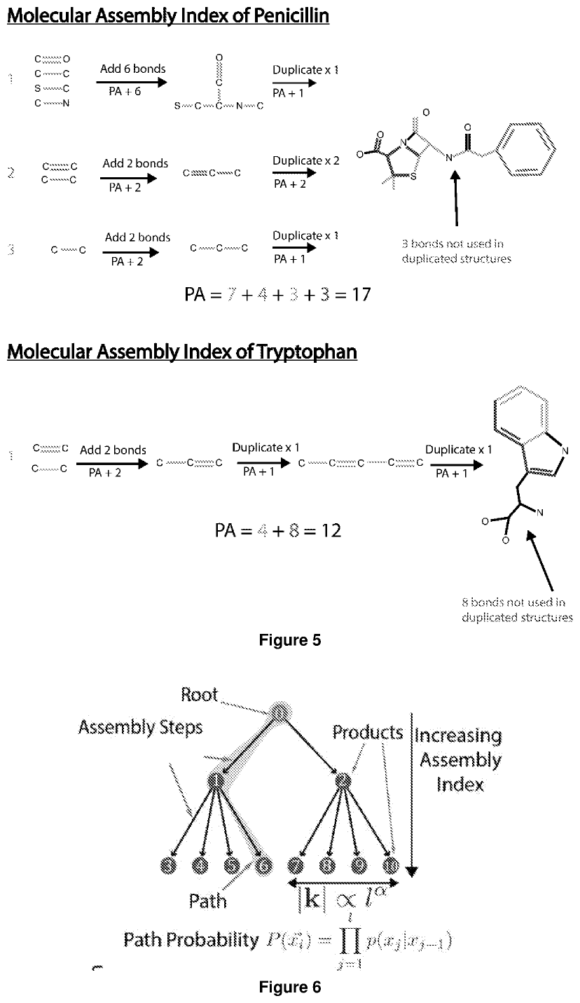

Several methods are used to reduce the computational expense of the calculation. Only substructures duplicated at least once are considered in the partitions, with bonds not in those substructures contributing 1 each to the MA. The order of searching through partitions is based on the size and multiplicity of the repeated substructures (e.g. three substructures of size 2, and two of size 4), and minimum/maximum MA values for such partitions can be calculated based on size alone. In this way, substructures can be searched in order of increasing minimum MA, which allows the algorithm to terminate when the minimum MA of a partition based on size/multiplicity is greater than or equal to best MA value found so far. A simplified flow-chart representation of the algorithm can be seen in FIGS. 2 to 4.

Random Decision Tree Model of Molecular Synthesis

In the present invention, MA is used distinguish molecules of biological or technological origin from abiotic chemical products. To accomplish this, a threshold MA index for a molecule must be determined above which any reliable synthesis must be due to biological or technological processes. To estimate a range of values for this threshold, the statistical properties of assembly pathways were explored by modelling the molecular assembly process as a random walk on directed trees.

In this model, the root of the tree corresponds to abiotically available precursors, while the number of leaves on the root correspond to the number of possible combinations of those precursors. Each node in the tree (besides the root) corresponds to molecules which could be synthesized from the available precursors. The depth of a given node (the shortest number of steps between it and the root) corresponds to the MA of that molecule, with those precursors, see FIG. 6. The breadth of the tree at depth i is labelled as kᵢ.

The statistical properties of the trees are controlled by adjusting the relative weights of the edges (to control the relative likelihood of forming one product over another) and the number of outgoing edges for each node (to control the total number of possible products).

The rates of chemical reactions can vary dramatically, often spanning several orders of magnitude. To account for this, edge weights (and therefore relative abiotic likelihoods of those reactions) are assigned that also span multiple orders of magnitude. Each edge weight is drawn from a distribution of the form wᵢ˜10^{u(0,h)}, where u(0, h) represents a uniform distribution between 0 and h such that h controls how many orders of magnitude the weights vary over. The weights are normalized such that the total weight of all out going edges is one, and therefore each probability has a value between zero and one.

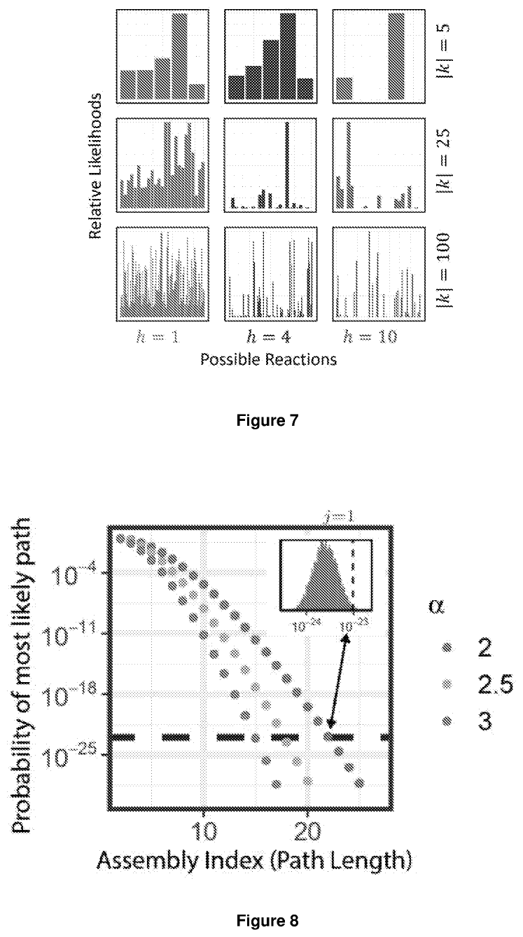

The effect of changing h is shown in FIG. 7. It can be seen that increasing h to give a more heterogeneous distribution increases the relative likelihood of the most likely path through the tree way. Thus, heterogeneous distributions funnel the probability towards a limited subset of all possible paths at each step. Our model uses h=4, such that probabilities of any given joining operation vary over four orders of magnitude. The inventor believe this captures the appropriate degree of bias.

The number of outgoing edges—which corresponds to the number of possible products—for each node in the trees grows as a function of the depth of the node. The rate of growth is modelled using a function of the form |k|∝l^{α}, where |k| is the number of outgoing edges, l is the depth of the node and α is a free parameter that controls how quickly the number of joining operations growths with the depth of the tree. The number of possible products in an assembly path is controlled by two different factors: the number of ways to pick two objects from the path to combine to form the next step, and the number of ways those two products themselves can be combined. The number of ways to pick two unique products must increase faster than linearly with the length of the path, since the paths recursively utilize previous steps. The number of possible products formed from the combination of two molecules grows linearly with the size of the molecule, since the bigger molecules have more atoms between which bonds can form. Given these fundamental constraints the model uses values of a between two and three, where two indicated the most conservative quadratic growth rate and three represents both factors growing super-linearly.

The model was used to calculate the probabilities of an assembly process resulting in a specific molecule as a function of the length of the assembly pathway to that molecule. These probabilities are calculated by starting at the root of the tree and multiplying all the edge weights for the path that leads from the root to the specific product. Since we are interested in the production of molecules in any abundance, we focused on identifying the probability of the most likely path in each tree, which we calculated exhaustively. For each path length (and therefore MA) the 99th percentile of those path probabilities was recorded, showing a conservatively high estimate of the probability of the most probable path. FIG. 8 shows the result of this analysis repeated over three different values of α. The probability of the most likely path drops dramatically as a function of the pathway length for all values of α, quickly dropping below one in a mole (10²³) for moderate path lengths with MA index greater than 15. This trend is observed although the formation of a narrow set of products is biased by using edge weights that are very heterogenous (h=4). This shows that the molecular assembly index tracks the specificity of a particular path through the combinatorically vast chemical space and supports our thesis that high MA molecules cannot form in detectable abundance through random and undirected processes.

MA in Theoretical Chemical Space

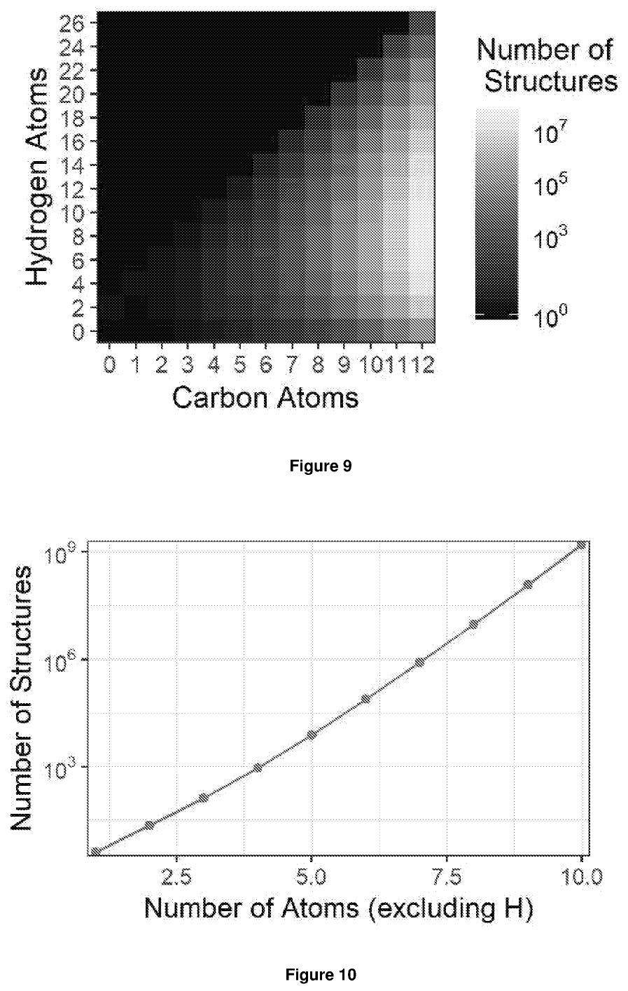

In order to estimate the size of a constrained subset of theoretical chemical space, the commercial software MOLGEN (Benecke) was used to enumerate all hydrocarbons with up to 12 carbon atoms, for all possible combinations of C and H atoms. As shown in FIG. 9, the number of possible structural isomers rises rapidly with the number of C atoms, peaking at C₁₂H₈ with approximately 47 million structural isomers.

The total number of possible structures containing only C, N, O, S, and H was also calculated. Calculations were performed up to the limit of 9 non-hydrogen atoms due to computational constraints. As shown in FIG. 10, the total number of structures is approximately 120 million. The number of structures for n non-hydrogen atoms was approximately (n+3)!/6, and assuming an increase at this rate would imply that the number of possible structures for 70 non-hydrogen atoms would be approximately 10¹⁰⁰, significantly higher than the estimated number of atoms in the observable universe. Conversely, the number of possible molecules in known chemical space initially increases as size increases from small molecules before dropping off as molecules become larger, less likely to be found in nature, and more difficult to synthesise. Using the Reaxys database (https://www.reaxys.com) as a proxy for known chemical space, the number of total molecules containing only C, N, O, S and H peaks at about 530 k molecules for 24 non-H atoms, before reducing to −12 k for 70 non-H atoms, with no substances having over 82 non-hydrogen atoms.

The disparity between the number of known molecules and the number of possible molecules suggests that novel ways of exploring chemical space are required to identify important molecules and processes amongst the chemical noise. Exploring the structure of chemical space using molecular assembly could help identify processes that increase chemical complexity and generate molecules for material design, drug discovery and processes critical to artificial life.

MA in Known Chemical Space

The MA for a subset of the Reaxys database was calculated. The subset contained comprising 2.5 million unique molecules over a molecular mass range of 0 to 800 Da.

These results show that for small molecules (mass <˜250 Da) the MA is strongly constrained by their mass. This is understandable because small molecules have limited compositional diversity and few structural asymmetries. Significantly, the MA of molecules with a mass greater than ˜250 Da appear to be significantly less constrained, indicating that they can display vastly more compositional and structural heterogeneity.

It appears that above 250 Da, the molecular weight of a molecule and its MA are effectively decoupled. To confirm that this was a true effect and not an artefact of the relatively low representation of high MA molecules in the data, the data was subsampled such that the molecular weight range was sampled uniformly. This subsampled data was used to generate

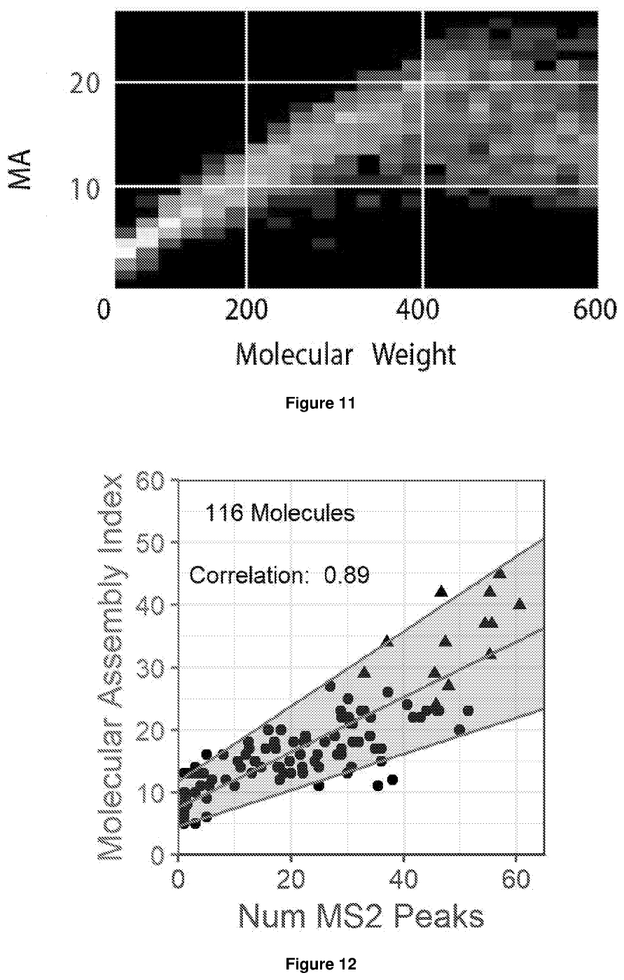

FIG. 11, which shows that MA is highly constrained by molecular weight for molecules with masses below 250 Da.

Calculated MA of Known Molecules

Using the above method, the MA for approximately 100 known small molecules was calculated. The results are set out in Table 1 ordered by decreasing calculated MA. Peptides are denoted using standard one-letter codes.

TABLE 1
Calculated MA for known small molecules

| | | | Molecular | |
|---|---|---|---|---|
| Descriptor | Molecules | Molecular Formula | Weights | MA |
| Peptide | YWHQNWYF | C₆₄H₆₈N₁₂O₁₅ | 1244.49 | 64 |
| Peptide | HWYQNWYF | C₆₄H₆₈N₁₂O₁₅ | 1244.49 | 63 |
| Peptide | HWYQNWYA | C₅₈H₆₄N₁₂O₁₅ | 1168.46 | 58 |
| Peptide | YWHQNWYW | C₆₆H₆₉N₁₃O₁₅ | 1283.5 | 53 |
| Peptide | QNWYF | C₃₈H₄₃N₇O₁₀ | 757.31 | 50 |
| Peptide | QNYWF | C₃₈H₄₃N₇O₁₀ | 757.31 | 50 |
| Peptide | QNWYA | C₃₂H₃₉N₇O₁₀ | 681.28 | 45 |
| Peptide | NWYF | C₃₃H₃₅N₅O₈ | 629.25 | 42 |
| Peptide | NYWF | C₃₃H₃₅N₅O₈ | 629.25 | 42 |
| Peptide | ASGNQSGV | C₂₇H₄₆N₁₀O₁₃ | 718.32 | 40 |
| Peptide | QNWYW | C₄₀H₄₄N₈O₁₀ | 796.32 | 40 |
| Peptide | FGSNQ | C₂₃H₃₃N₇O₉ | 551.23 | 37 |
| Peptide | NWYA | C₂₇H₃₁N₅O₈ | 553.22 | 37 |
| Peptide | WYF | C₂₉H₂₉N₃O₆ | 515.21 | 34 |
| Peptide | YWF | C₂₉H₂₉N₃O₆ | 515.21 | 34 |
| Peptide | NWYW | C₃₅H₃₆N₆O₈ | 668.26 | 32 |
| Peptide | FGSN | C₁₈H₂₅N₅O₇ | 423.18 | 29 |
| Peptide | WYA | C₂₃H₂₅N₃O₆ | 439.17 | 29 |
| Peptide | GSGNQ | C₁₆H₂₇N₇O₉ | 461.19 | 27 |
| Pharmaceutical | Ceftiofur | C₁₉H₁₇N₅O₇S | 523.03 | 27 |
| Pharmaceutical | Sildenafil | C₂₂H₃₀N₆O₄S | 474.20 | 26 |
| Pharmaceutical | GW843682 | C₂₂H₁₈F₃N₃O₄S | 477.10 | 25 |
| Agrochemical | Primisulfuron-methyl | C₁₅H₁₂F₄N₄O₇S | 468.04 | 24 |
| Peptide | WYW | C₃₁H₃₀N₄O₆ | 554.22 | 24 |
| | Folic Acid | C₁₉H₁₉N₇O₆ | 441.14 | 23 |
| Pharmaceutical | Aztreonam | C₁₃H₁₇N₅O₈S₂ | 435.05 | 23 |
| Pharmaceutical | Cefixime | C₁₆H₁₅N₅O₇S₂ | 453.04 | 23 |
| | (3′,9S)-6′-Methoxy- | C₂₇H₂₉ClN₂O₃ | 464.19 | 23 |
| | 10,11-dihydrocinchonan- | | | |
| | 9-yl 4-chlorobenzoate | | | |
| Pharmaceutical | Cefpodoxime | C₁₅H₁₇N₅O₆S₂ | 427.06 | 23 |
| Pharmaceutical | Ketoconazole | C₂₆H₂₈Cl₂N₄O₄ | 530.15 | 23 |
| Pharmaceutical | SR1001 | C₁₅H₁₃F₆N₃O₄S₂ | 477.03 | 23 |
| Pharmaceutical | GW8510 | C₂₁H₁₅N₅O₃S₂ | 449.06 | 23 |
| Agrochemical | Cyclosulfamuron | C₁₇H₁₉N₅O₆S | 421.11 | 22 |
| Agrochemical | Oxasulfuron | C₁₇H₁₈N₄O₆S | 406.09 | 22 |
| Pharmaceutical | Paliperidone | C₂₃H₂₇FN₄O₃ | 426.21 | 22 |
| | Ethyl 6-methyl-2-({oxo[(2E)-2-(4- | C₂₀H₂₂N₄O₄S | 414.14 | 22 |
| | pyridinylmethylene)hydrazino]acetyl}amino)- | | | |
| | 4,5,6,7-tetrahydro-1-benzothiophene- | | | |
| | 3-carboxylate | | | |
| Bioactive | 8-(4-Chlorophenylthio)adenosine | C₁₆H₁₄ClN₅NaO₆PS | 493.00 | 22 |
| | 3′,5′-cyclic monophosphate | | | |
| | sodium salt | | | |
| Bioactive | Necrostatin-5 | C₁₉H₁₇N₃O₂S₂ | 383.08 | 21 |
| Bioactive | Pirenperone | C₂₃H₂₄FN₃O₂ | 393.19 | 21 |
| Pharmaceutical | SCH-58261 | C₁₈H₁₅N₇O | 345.13 | 20 |
| Bioactive | CITCO | C₁₉H₁₂Cl₃N₃OS | 434.98 | 20 |
| Bioactive | BAY 41-2272 | C₂₀H₁₇FN₆ | 360.15 | 20 |
| Pharmaceutical | Capecitabine | C₁₅H₂₂FN₃O₆ | 359.15 | 19 |
| Pharmaceutical | Tenoxicam | C₁₃H₁₁N₃O₄S₂ | 337.02 | 19 |
| Bioactive | Necrostatin-7 | C₁₆H₁₀FN₅OS₂ | 371.03 | 19 |
| Dye | 1,3,3-Trimethylindolino-6′- | C₁₉H₁₈N₂O₃ | 322.13 | 18 |
| | nitrobenzo-pyrylospiran | | | |
| | Boc-Lys(Nicotinoyl)-OH | C₁₇H₂₅N₃O₅ | 351.18 | 18 |
| | 5-[3-(4-Fluorophenyl)-1-phenyl- | C₁₈H₁₃FN₄O₂ | 336.10 | 18 |
| | 1H-pyrazol-4-yl]-2,4- | | | |
| | imidazolidinedione(5-(1,3- | | | |
| | diaryl-1H-pyrazol-4-yl)hydantoin, | | | |
| | 5-[3-(4-fluorophenyl)-1- | | | |
| | phenyl-1H-pyrazol-4-yl]-2,4- | | | |
| | imidazolidinedione) | | | |
| Pharmaceutical | Ampicillin sodium salt | C₁₆H₁₈N₃NaO₄S | 371.09 | 18 |
| Synthetic | Fmoc N-hydroxysuccinimide ester | C₁₉H₁₅NO₅ | 337.10 | 18 |
| intermediate | | | | |
| Pharmaceutical | Merafloxacin | C₁₉H₂₃F₂N₃O₃ | 379.17 | 18 |
| Pharmaceutical | Cefalexin | C₁₆H₁₇N₃O₄S | 347.09 | 18 |
| Agrochemical | Prochloraz | C₁₅H₁₆Cl₃N₃O₂ | 375.03 | 18 |
| Bioactive | LY-171,883 | C₁₆H₂₂N₄O₃ | 318.17 | 17 |
| Biomolecule | 2′-Deoxyadenosine | C₁₀H₁₄N₅O₆P | 331.07 | 17 |
| | 5′-monophosphate | | | |
| Synthetic | CDS014803 | C₁₄H₁₇N₃O₃S | 307.10 | 17 |
| intermediate | | | | |
| Bioactive | IM12 | C₂₂H₂₀FN₃O₂ | 377.15 | 17 |
| Synthetic | FMOC Leucine | C₂₁H₂₃NO₄ | 353.16 | 17 |
| intermediate | | | | |
| Bioactive | (6-[1-(3,5,5,8,8-Pentamethyl- | C₂₄H₂₉NO₂ | 363.22 | 17 |
| | 5,6,7,8-tetrahydro-2- | | | |
| | naphthalenyl)cyclopropyl]nicotinic acid) | | | |
| | 3-(1,1-Dioxido-4-thiomorpholinyl)- | C₁₄H₁₉NO₅S | 313.10 | 16 |
| | 3-(4-methoxyphenyl)propanoic acid | | | |
| Pharmaceutical | Domperidone | C₂₂H₂₄ClN₅O₂ | 425.16 | 16 |
| | 1-(3-Nitropyridin-2-yl)pyrrolidine- | C₁₀H₁₁N₃O₄ | 237.07 | 16 |
| | 2-carboxylic Acid | | | |
| | CDS016302 | C₉H₈F₃N₃OS | 263.03 | 16 |
| Pharmaceutical | Flubendazole | C₁₆H₁₂FN₃O₃ | 313.09 | 16 |
| | (5,7-dimethyl-[1,2,4]triazolo[4,3- | C₉H₁₀N₄O₂S | 238.05 | 16 |
| | a]pyrimidin-3-ylsulfanyl)- | | | |
| | acetic acid | | | |
| Pharmaceutical | Leflunomide | C₁₂H₉F₃N₂O₂ | 270.06 | 16 |
| Pharmaceutical | Fluconazole | C₁₃H₁₂F₂N₆O | 306.10 | 15 |
| Bioactive | Rolipram | C₁₆H₂₁NO₃ | 275.15 | 15 |
| Synthetic | Fmoc L-Alanine | C₁₈H₁₇NO₄ | 311.12 | 15 |
| intermediate | | | | |
| Pharmaceutical | Minoxidil sulfate salt | C₉H₁₅N₅O₄S | 289.08 | 15 |
| Pharmaceutical | Clozapine | C₁₈H₁₉ClN₄ | 326.13 | 15 |
| | 2-(5-Methyl-2-thienyl)-4- | C₁₅H₁₁NO₂S | 269.05 | 14 |
| | quinolinecarboxylic acid | | | |
| Pharmaceutical | Cinnarizine | C₂₆H₂₈N₂ | 368.23 | 14 |
| Dye | Methylene blue | C₁₆H₁₈ClN₃S | 319.09 | 14 |
| Dye | Quinoline Yellow | C₁₈H₁₁NO₂ | 273.08 | 14 |
| Dye | Thioflavin T | C₁₇H₁₉ClN₂S | 318.10 | 14 |
| Bioactive | UPF-1069 | C₁₇H₁₃NO₃ | 279.09 | 14 |
| | 7-(2-Hydroxypropyl)theophylline | C₁₀H₁₄N₄O₃ | 238.11 | 14 |
| Pharmaceutical | Dipyridamole | C₂₄H₄₀N₈O₄ | 504.32 | 14 |
| | 5-Amino-1-(3-chlorophenyl)- | C₁₀H₇ClN₄ | 218.04 | 14 |
| | 1H-pyrazole-4-carbonitrile | | | |
| Nucleoside | Thymidine | C₁₀H₁₄N₂O₅ | 242.09 | 13 |
| Liquid crystal | N-(4-Methoxybenzylidene)- | C₁₈H₂₁NO | 267.16 | 13 |
| | 4-butylaniline | | | |
| Biomolecule | Kinetin | C₁₀H₉N₅O | 215.08 | 13 |
| | 6-[4-(Methylsulfonyl)phenyl]- | C₁₃H₁₁NO₃S | 261.05 | 13 |
| | 2-pyridinecarboxaldehyde | | | |
| Biomolecule | D(+)-Biotin | C₁₀H₁₆N₂O₃S | 244.09 | 13 |
| Pharmaceutical | Hydrochlorothiazide | C₇H₈ClN₃O₄S₂ | 296.96 | 13 |
| | Triethoxy-3-(2-imidazolin- | C₁₂H₂₆N₂O₃Si | 274.17 | 13 |
| | 1-yl)propylsilane | | | |
| Nucleoside | Cytidine | C₉H₁₃N₃O₅ | 243.09 | 13 |
| | 2-Hydroxy-5-nitrobenzaldehyde | C₇H₅NO₄ | 167.02 | 12 |
| | 4,4′,4″-Tri-tert-Butyl- | C₂₇H₃₅N₃ | 401.28 | 12 |
| | 2,2′:6′,2″-terpyridine | | | |
| Amino acid | Tryptophan | C₁₁H₁₂N₂O₂ | 204.09 | 12 |
| Pharmaceutical | Dienogest | C₂₀H₂₅NO₂ | 311.19 | 12 |
| | 3-(3-Methoxyphenyl)thiomorpholine | C₁₁H₁₅NOS | 209.09 | 11 |
| Curing agent | 1,1′-(Methylenedi-4,1- | C₂₁H₁₄N₂O₄ | 358.1 | 11 |
| | phenylene)bismaleimide | | | |
| | 2-(2-Thienyl)isonicotinic acid | C₁₀H₇NO₂S | 205.02 | 11 |
| Pharmaceutical | Chlorquinaldol | C₁₀H₇Cl₂NO | 226.99 | 11 |
| C₁₁H₁₂O₅ | Sinapinic acid | C₁₁H₁₂O₅ | 224.07 | 11 |
| Amino acid | Arginine | C₆H₁₄N₄O₂ | 174.11 | 10 |
| Amino acid | Tyrosine | C₉H₁₁NO₃ | 181.07 | 10 |
| | Methyl adamantane-1-carboxylate | C₁₂H₁₈O₂ | 194.13 | 9 |
| | 5-Aminoisophthalic acid | C₈H₇NO₄ | 181.04 | 9 |
| Pharmaceutical | Sulfanilamide | C₆H₈N₂O₂S | 172.03 | 9 |
| Amino acid | Methionine | C₅H₁₁NO₂S | 149.05 | 8 |
| Amino acid | Proline | C₅H₉NO₂ | 115.06 | 8 |
| Metabolite | Aconitic Acid | C₆H₆O₆ | 174.02 | 7 |
| Amino acid | Asparagine | C₄H₈N₂O₃ | 132.05 | 7 |
| Amino acid | Lysine | C₆H₁₄N₂O₂ | 146.11 | 7 |
| Amino acid | Cysteine | C₃H₇NO₂S | 121.02 | 6 |
| Metabolite | Ketoglutaric Acid | C₅H₆O₅ | 146.02 | 6 |
| Metabolite | Oxaloacetic Acid | C₄H₄O₅ | 132.01 | 6 |
| Amino acid | Threonine | C₄H₉NO₃ | 119.06 | 6 |
| Curing agent | 1-(2-Aminoethyl)piperazine | C₆H₁₅N₃ | 129.13 | 5 |
| Amino acid | Serine | C₃H₇NO₃ | 105.04 | 5 |
| Metabolite | Succinic Acid | C₄H₆O₄ | 118.03 | 5 |

Experimental MA

Below, we describe examples of determining molecular assembly index using tandem mass spectrometry.

Mass Spectrometry Workflow

All samples were analysed by tandem mass spectrometry in an Orbitrap Fusion Lumos Tribrid mass spectrometer (Thermo, San Jose, Calif., USA). Molecules analysed for the standard curve calculation were introduced to the mass spectrometer via the Advion Nanomate (Ithaca, N.Y., USA). Samples of 15 μI were injected onto an emitter with a +1.2 KV voltage applied, the gas on the nanomate was set to 40 psi. Samples were analysed for 6 mins where a Single Ion Monitoring (SIM) scan for a molecules exact mass was performed followed by a fragmentation event (MS2). This ensured fragmentation data was collected for the targeted analyte and not any potential contamination. The fragmentation method was HCD with fragmentation energies set at 45% for the first 3 mins and 35% for mins 3 to 6. The isolation window for MS2 fragmentation selection was set to 0.5 Da, the resolution of the SIM scan was 240,000 and the resolution of the MS2 scans was 30,000.

MS data was converted into mzML files using MS Convert (Adusumilli. & Mallick) and the mzML files was converted to a Json peak list, with all MS1 peaks collected for each m/z over the 6 minutes analysis being merged. Spectra with maximum intensity under 50000 were discarded, and for those remaining all peaks within 0.01 Da were merged. All MS2 peaks not present in at least 25% of MS2 spectra from the corresponding MS1 parent were disregarded. Any peak within ±1.0 Da from an adjacent peak was merged, reducing the over count of ions which differ only by one hydrogen atom. The remaining MS2 peaks were counted, and this number was used with the calculated MA of the molecular graph associated with the MS1 peak to generate the correlation.

Environmental samples were analysed under the same ionisation conditions. However, the mass spectrometer was run with a Data Dependent Acquisition (DDA) method which fragmented the 15 most intense ions, using a dynamic exclusion of 30 s if the analyte was present twice in 10 s, with a mass range of 300-500 m/z. Given the number of ions in the complex environmental samples, filtering was also used to remove peaks from the MS2 spectra with an intensity below 10% relative to the highest observed peak in that spectra. All other parameters were as above. In the analysis of the complex environmental samples, the inventor noticed that co-fragmentation resulted in excessively high numbers of MS2 peaks by effectively merging different MS1 parent ions into the same MS2 spectra. Thus, after counting the number of MS2 peaks for each selected parent ion, the MS1 spectra was checked for peaks within 0.5 Da of the parent ion. The total number of MS2 peaks was divided by the number of MS1 peaks found within 0.5 Da of the parent mass. This accounts for the co-fragmentation patterns because it divides the number of MS2 peaks across the total number of identified unique ions in the collision cell during the fragmentation. This method was used in all samples and was found not to affect the previous results for single ions.

Single Molecule Sample Analysis

The inventor collected MS2 spectra for 116 small molecules and peptides for which the MA had been calculated (see Table 1). The inventor compared the number of MS2 peaks in the spectra to the calculated MA for all molecules. The results are shown in FIG. 12, where each point represents a unique molecule. The analysis demonstrates a linear relationship, with a correlation of 0.89, between the number of MS2 peaks generated by a fragmented ion and its MA. The linear relationship was fit using quantile regression, where the upper line is fit with τ=0.95, the middle line is the median fit with τ=0.5, and the lower line is the fit with τ=0.05, such that the shaded region shows the uncertainty in the relationship with 90% confidence, while the middle line shows the expected fit.

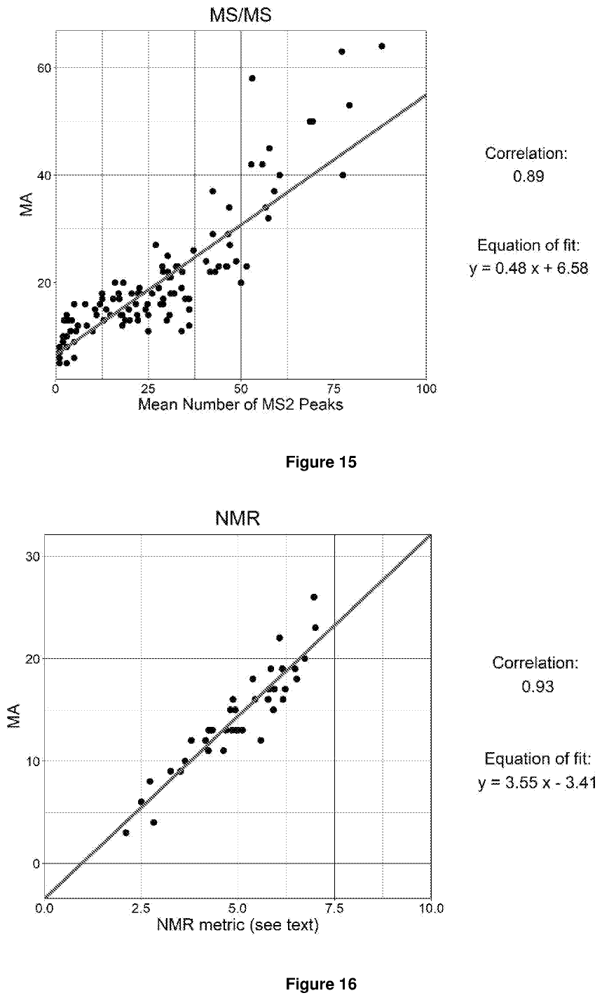

FIG. 15 is plot of the same results on a log scale (base 2). The observed linear correlation between the number of peaks in the MS2 spectrum and the MA value of a molecule has a slope of 0.48 and an off-set of 6.58.

Environmental Sample Collection

Having established the ability to experimentally determine the MA of molecules using tandem MS, the inventor sought to directly test the hypothesis that high MA molecules can only be produced by living systems. Thus, several mixtures, including those sourced from biological, abiotic, and dead sources were prepared and analyzed. Each sample was prepared with a similar procedure with the only significant differences arising due to the different nature of the samples. The details regarding the preparation of those samples are listed below.

Yeast: A solution of sucrose was added to 1 g of commercially available baker's yeast and allowed to activate at room temperature overnight. On observation of carbon dioxide bubbles, the yeast was centrifuged at 13,000 rpm for 10 mins. The supernatant was discarded, and the pellet was split into 4 samples. One sample was labelled native and 1 mL of methanol was added followed by 30 mins sonication. The other three samples were analysed by Thermo gravimetric analysis (TGA) at three different temperatures 200° C., 400° C. and 600° C. The charred samples were then extracted in methanol, and all four samples were filtered prior to mass spec analysis.

E. coli: Escherichia Coli MG1655 was purchased from DSMZ (Germany). Bacteria cells were grown overnight in a 50 mL lysogeny broth (LB) media at 37° C. and 250 rpm until O.D. of 0.6 was achieved. A 5:100 dilution in fresh media was incubated overnight at 3° C. and 250 rpm and harvested when O.D. was 1.8-2.0. Bacterial culture was then centrifuged for 10 minutes to form a cells pellet which was washed twice with 50-100 mL of ice-cold water. After that, the wet pellet was dissolved in water to make a final concentration of ca. 1 g/mL. Mechanical cell lysis using bead beating method was used to avoid any chemical or enzymatic interference. In a beat beating tube, 500 μL of cell solution was mixed with 500 μL of water and was run on the beat beater machine for 30 seconds followed by incubation on ice for another 30 seconds. This process was repeated 10 times. Samples were centrifuged at 4° C. for 3 min before extracting the cell supernatant and centrifuging the samples again for 60 minutes. The resulted supernatant was collected and stored at −80° C. for further analysis.

Urine: Urine was mixed 50:50 with 2 M urea, 10 mM NH₄OH and 0.02% SDS. Samples were filtered with Centristat, 20 kDa cutoff, (Satorius, Göttingen, Germany). The filtrate was desalted in a PD-10 column (GE Healthcare Bio Sciences, Uppsala, Sweden). The processed urine was dried and stored at 4° C. before use. The Standard Urine sample was reconstituted in 500 μL H₂O before injection into the mass spectrometer.

Rock and Soil Samples: Coal, Serpentine, Sandstone, Limestone, Granite, Quartz and Clay were separately crushed in a rock crusher and sieved through a series of sieves. The fraction from the <0.25 mm sieve was collected. Rocks were supplied by Richard Tayler Minerals (Surrey UK). Rock dust (1 mg) was submerged in 1 mL of MeOH overnight at room temperature, centrifuged at 13,000 rpm for 10 mins and the resultant supernatant removed and filtered through Wattman paper. The eluent was loaded onto a 96 well plate and analyzed by mass spectrometry.

Beer: Home brewed beer courtesy of Dr James Ward Taylor was mixed 50:50 with MeOH. Samples were then loaded onto a 96 well plate and injected into the mass spectrometer.

Dipeptides: Dipeptides (1 mg) were weighed and reconstituted in 50:50 MeOH:H2O. Samples were loaded onto a 96 well plate and injected into the mass spectrometer.SI-6.7

Whisky: Whisky was donated from members of the Cronin research group at the University of Glasgow as well as The Jar Troon Whisky Specialists, Troon Ayrshire. Samples were diluted 1:50 with LC-MS grade H₂O before loaded onto a 96 well plate and injected and analysed using the same methods as previously described.

Mixed Molecule Sample Analysis

MS2 spectra were collected from a wide variety of mixtures, including biological samples such as: E. coli lysates, yeast cultures, urinary peptides, and fermented beverages (home brewed beer and Scottish Whisky), as well as abiotic samples, including: dipeptides, Miller-Urey mixtures, terrestrial rocks, and a carbonaceous meteorite.

The above analysis of the Reaxys database indicated molecules having a mass above 250 Da take on a diverse set of MA values. Therefore, in the mixed samples, MS1 peaks in the m/z range of 300-500 were selected for fragmentation and MS2 analysis.

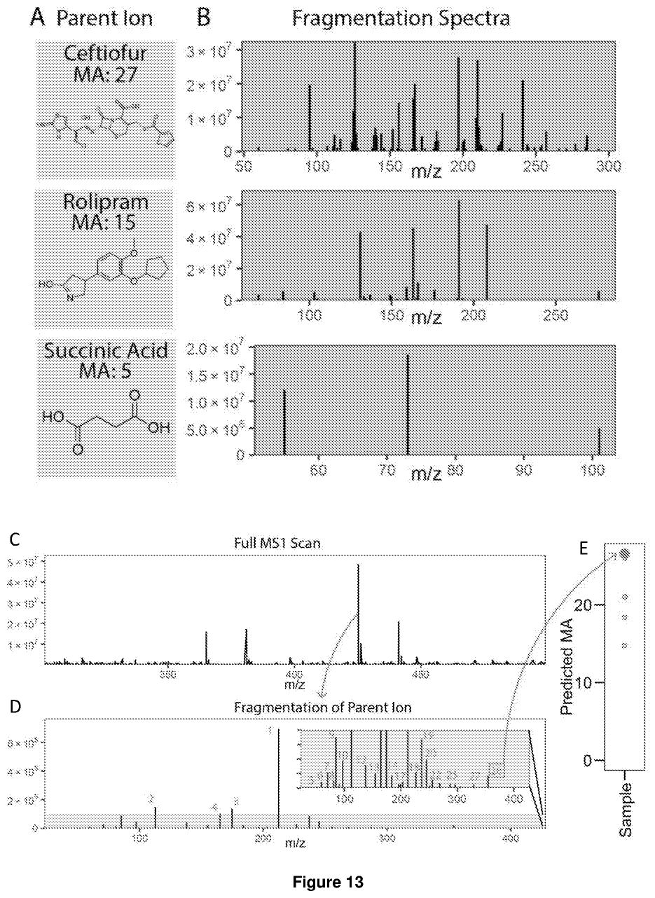

Fragmenting the most intense MS1 peaks permitted the collection of many distinct MS2 spectra from the mixtures. The number peaks in each MS2 spectra was counted and the observed correlation from the single-molecule analysis was used to predicted the MA of the different ions in the mixtures. FIGS. 13C to 13E illustrate this workflow, indicating the selected peak in the MS1 spectra (C), the associated MS2 spectra (D) with the peaks counted, with the inset zoomed in on the same data to show the lower intensity peaks, and the predicted MA for that MS2 spectra and others from the same mixture (E).

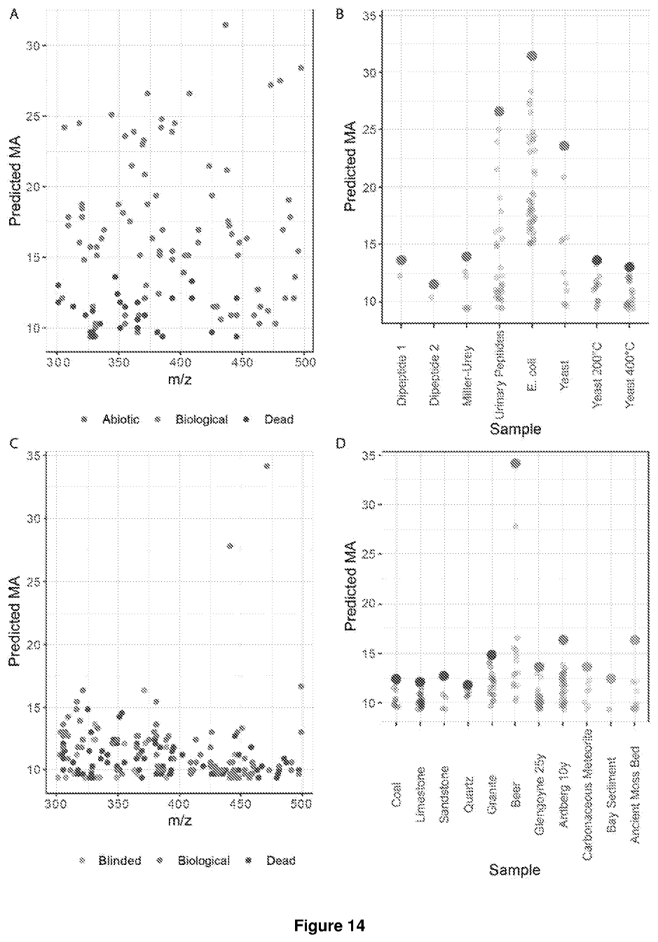

FIGS. 14(A) and (B) show the results for mixtures prepared in the lab, while FIG. 14(C) and (D) show the results for mixtures collected from the environment. FIG. 14(A) shows the predicted MA of all ions in the 300-500 m/z range against their parent mass, coloured by their source. FIG. 14(B) shows the predicted MA for each sample separately, with the highest observed MA bolded and the lower values faded out.

These results demonstrate that it is possible to identify the living systems by looking for mixtures with an MA greater than a certain threshold. In the analysis presented here, it appears that only living samples produced MA measurement above about 15. Importantly, this measurement does not imply that samples with a maximum MA below 15 are non-living. Indeed, many samples made or altered by living systems failed to generate an MA above this threshold such as the Bay Sediment and some of the Scottish whisky. These examples represent false negatives. Not all molecules produced by living processes have high MA.

Indeed, complex mammals regularly produce 002. However, all high MA molecules are produced by living (or technological) processes. This is critical as it implies that looking for high (specifically, greater than 15) MA values in mixtures is an agnostic way to search for living systems.

NMR Measurement

The theoretical ¹H and ¹³C NMR spectra were generated for each molecular structure. The number of different peaks in the ¹H and ¹³C NMR were summed, and this value was weighted against the number of C and CH moieties per molecule. The results are shown in FIG. 16.

A linear correlation is observed with a fit of 0.93. The slope is 3.55 and the off-set is −3.41. This give a preliminary indication that NMR spectroscopy could be useful in PA based life detection systems as one of a suite of analytical tools. It could be of particular use where a non-destructive analytical technique is required.

IR Measurement

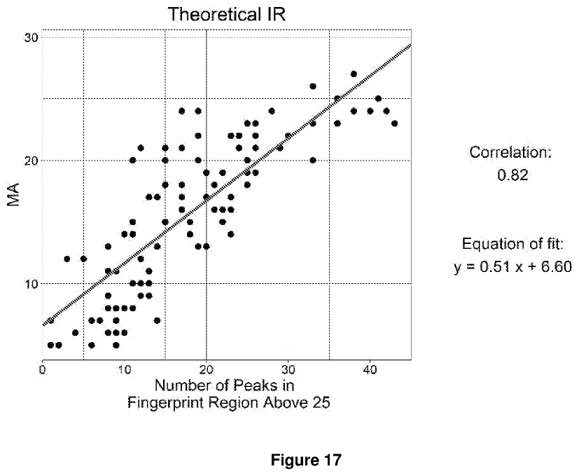

IR data were modelled for a total of 101 molecules and a count was extracted of the number of peaks within the fingerprint region. The correlation between the number of peaks in the fingerprint region above an intensity threshold is shown in FIG. 17. A linear relationship was observed with a fit of 0.82. The slope was 0.51 and the off-set was 6.60. This give a preliminary indication that IR spectroscopy could be useful in PA based life detection systems as one of a suite of analytical tools. It could be of particular use where a remote or non-destructive analytical technique is required.

### Abbreviations

Da Daltons

MA (Split-branch) molecular assembly index

### REFERENCES

A number of publications are cited above in order to more fully describe and disclose the invention and the state of the art to which the invention pertains. Full citations for these references are provided below. The entirety of each of these references is incorporated herein.
ADUSUMILLI, R. & MALLICK, P., 2017, Vol. 1550, Humana Press.
ALLU, T. K. & OPREA, T. I., 2005, Journal of Chemical Information and Modeling, Vol. 45, pp. 1237-1243.
ANBAR, A. D., 2004, Earth and Planetary Science Letters, Vol. 217, pp. 223-236.
BALABAN, A. T., 1983, Pure and Applied Chemistry, Vol. 55 pp. 199-206.
BARONE, R. & CHANON, M., 2001, Journal of Chemical Information and Computer Sciences, Vol. 41, pp. 269-272.
BENECKE, C. et al., 1997, Fresenius' Journal of Analytical Chemistry, Vol. 359, pp. 23-32.
BENNER, S. A., 2017, Astrobiology, Vol. 17, pp. 840-851.
BERTZ, S. H., 1981, Journal of the American Chemical Society, Vol. 103, pp. 3599-3601.
BONCHEV, D. & TRINAJSTIĆ, N., 1977, The Journal of Chemical Physics, Vol. 67, pp. 4517-4533.
BÖTTCHER, T., 2016, Journal of Chemical Information and Modelling, Vol. 56, pp. 462-470.
BRESLOW, R. & LEVINE M. S., 2006, Proceedings of the National Academy of Sciences, Vol. 103, pp. 12979-12980.
COLEY, C. W., et al., Vol. 58, pp. 252-261.
DEEPAISARN, S. et al., 2018, Bioinformatics, Vol. 34, pp. 1001-1008.
DES MARAIS, D. J. & WALTER, M. R., 1999, Annual Review of Ecology and Systematics, Vol. 30, pp. 397-420.
DES MARAIS, D. J. et al., 2008, Astrobiology, Vol. 8, pp. 715-730.
GEORGIOU, C. D. & DEAMER, D. W., 2014, Astrobiology, Vol. 14, pp. 541-549.
HAAN, M. H. et al., 2001, Journal of Chemical Information and Computer Sciences, Vol. 41, pp. 856-864.
HELLER, S. R. et al., 2015, Vol. 7, No. 23.
LI, J. & EASTGATE, M. D., 2015, Organic & Biomolecular Chemistry, Vol. 13, pp. 7164-7176.
MACDERMOTT, A. J. et al., 1996, Planetary and Space Science, Vol. 44, pp. 1441-1446.
MARSHALL, S. M. et al., 2017, Philosophical Transactions of the Royal Society A: Mathematical, Vol. 375, No. 20160342.
MARSHALL, S. M., et al., 2019, arXiv e-prints, arXiv:1907.04649.
MINOLI, D., 1975, Atti della Accademia Nazionale dei Lincei, Vol. 59, pp. 651-661.
NEVEU, M. et al., 2018, Astrobiology, Vol. 18, pp. 1375-1402.
RANDĆ, M. & PLAŠIĆ, D., 2002, Croatica Chemica Acta 75, 107-116 (2002).
RÜCKER, G. & RÜCKER, C. 2000, Journal of Chemical Information and Computer Sciences, Vol. 40, pp. 99-106.
RÜCKER, G. & RÜCKER, C. 2001, Journal of Chemical Information and Computer Sciences, Vol. 41, pp. 1457-1462.
SEAGER, S. et al. 2005, Astrobiology Vol. 5, pp. 372-390.
SCHWIETERMAN, E. W. et al., 2018, Astrobiology, Vol. 18, pp. 663-708.
SHERIDAN, R. P. et al., 2014, Journal of Chemical Information and Modeling, Vol 54, pp. 1604-1616.
VON KORFF, M. & SANDER, T., 2019, Scientific Reports, Vol. 9, No. 967.
WALKER, S. I. et al., 2018, Astrobiology, Vol. 18, pp. 779-824.
ZHANG, Q. et al., 2016, Journal of Chemometrics, Vol. 30, pp. 70-74.

1. A method for estimating the molecular complexity of a sample, the method comprising:
(a) performing one of MS/MS, NMR or IR on a sample;
(b) determining the unique peaks in the resulting MS2 spectrum for a parent ion in the MS1 spectrum, NMR spectrum, or IR spectrum; and
(c) calculating the molecular assembly index of the sample based on the number of unique peaks in the resulting MS2, NMR or IR spectrum.

2. The method of claim 1, wherein the method comprises:
(a) performing MS/MS on the sample;
(b) determining the unique peaks in an MS2 spectrum for a parent ion in the MS1 spectrum; and
(c) calculating the molecular assembly index of the sample based on the number of unique peaks in the MS2 spectrum.

3. The method of claim 2, wherein step (a) comprises selecting a parent ion in the MS1 spectrum having a mass of 250 Da or more.

4. The method of claim 2, wherein step (a) comprises selecting a parent ion in the MS1 spectrum having a mass of 1000 Da or less.

5. The method of claim 2, wherein step (b) comprises disregarding peaks in the MS2 spectrum having a maximum intensity of 0.5% or less than the highest recorded intensity in the MS1 spectrum.

6. The method of claim 2, wherein step (b) comprises merging all peaks within ±0.01 Da in the MS2 spectrum.

7. The method of claim 2, wherein step (b) comprises merging any peak within ±1.0 Da of an adjacent peak in the MS2 spectrum.

8. The method of claim 2, wherein:
step (a) comprises recording multiple MS2 spectra for a parent chosen ion in the MS1 spectrum; and
step (b) comprises disregarding all peaks not present in at least 25% of the MS2 spectra from the chosen parent ion.

9. The method of claim 2, wherein the molecular assembly index is calculated by scaling the number of unique peaks in the MS2 spectrum by a magnitude (m), optionally wherein the magnitude (m) is in the range 0.3 to 0.7.

10. The method of claim 2, wherein the molecular assembly index is calculated by adjusting the number of unique peals in the MS2 spectrum by an off-set (c), optionally wherein the off-set (c) is in the range 4 to 9.

11. The method of claim 2, wherein the molecular assembly index (MA) is calculated by scaling the number of unique peaks in the MS2 (x_{MS2}) spectrum using the equation:
MA=0.48(x_{MS2})+6.58,

12. The method of claim 2, wherein step (a) comprises selecting between 5 and 20 of the most intense peaks in the MS1 spectrum and recording MS2 spectra for each peak.

13. The method of claim 2, wherein step (a) comprises excluding parent ions appearing twice in a set time interval, for example using dynamic exclusion, optionally wherein a dynamic exclusion of 30 to 90 seconds is used for parent ions appearing twice in 10 seconds.

14. The method of claim 2, wherein:
(i) step (b) comprises disregarding all peaks with a relative intensify below 10% of the highest recorded intensity in each MS2 spectrum; and/or
(ii) step (b) comprises dividing the number of peaks in the MS2 spectrum by the number of peaks in the MS1 spectrum within 0.5 Da of the parent ion.

15. (canceled)

16. The method of claim 2, wherein step (c) comprises calculating the molecular assembly index of the sample from the parent ion having the largest number of unique peaks in the MS2 spectrum.

17. A method for the detection of life, the method comprising:
(a) estimating the molecular complexity of a sample according to the method of claim 1; and
(b) comparing the calculated molecular assembly index of the sample to a threshold value.

18. The method of claim 17, wherein the sample is a sample of extra-terrestrial material or the sample is a sample of terrestrial material, such as terrestrial soil, water, ice or rock.

19. (canceled)

20. A method for identifying a candidate pharmaceutical or agrochemical molecule, the method comprising:
(a) estimating the molecular complexity of a sample in a molecular library using the method of claim 1; and
(b) selecting a sample having a molecular assembly index greater that a threshold value as a candidate agrochemical or pharmaceutical molecule.

21. The method of claim 20, wherein the method comprises:
(c) separating the sample constituents; and optionally
(d) screening the sample constituents for biological activity.

22. The method of claim 20, wherein the sample is an extract from a biological source, such as an extract from a fermentation broth or a plant extract.
